(19) United States
(12) Patent Application Publication
(10) Pub. No.: US 2006/0234990 A1
(43) Pub. Date: Oct. 19, 2006

(54) Compositions of boswellic acids derived from Boswellia serrata gum resin, for treating lymphoproliferative and autoimmune conditions

(75) Inventors: Muhammed Majeed, Piscataway, NJ (US); Vladimir Badmaev, Piscataway, NJ (US)

Correspondence Address: ARENT FOX PLLC, 1050 CONNECTICUT AVENUE, N.W., SUITE 400, WASHINGTON, DC, 20036, US

(73) Assignee: SAMI LABS LTD.

(21) Appl. No.: 11/417,155

(22) Filed: May 4, 2006

Related U.S. Application Data
(62) Division of application No. 09/926,424, filed on Jun. 28, 2002.

(51) Int. Cl. A61K 31/56; A61K 31/20

(52) U.S. Cl. 514/170; 514/559

(57) ABSTRACT

A method of treatment of lymphoproliferative and autoimmune disorders with a new composition of four boswellic acids including β-boswellic acid, 3-O-acetyl-β-boswellic acid, 11-keto-β-boswellic acid, and 3-O-acetyl-11-keto-β-boswellic acid. Boswellic acids of invention have been obtained in a novel industrial process from the gum resin of Boswellia serrata tree, providing standardized composition which inhibits DNA, RNA and protein synthesis of the target cell without cytotoxic effects. The composition of invention provides advantage of irreversible cytostatic therapy, equivalent to biological effects of a cytotoxic therapy without killing body cells.

### BACKGROUND OF THE INVENTION

The present invention concerns new compositions of boswellic acids, methods of using the compositions or individual boswellic acids to treat lymphoproliferative and autoimmune conditions, and two new methods of isolating the new compositions.

Boswellia serrata N.O. Burseraceae) is a large, branching, deciduous tree which grows abundantly in the dry, hilly parts of India. It is known as “Dhup”, Indian Frankincense or Indian Olibanum. The gum resin exudate of Boswellia serrata, known in the vernacular as “Salai guggal”, has been used in the Ayurvedic system of medicine for the management of rheumatism, respiratory diseases, and liver disorders. The major use of Boswellia serrata in contemporary medicine is as an anti-arthritic and anti-inflammatory pharmacological agent.

The active principles of the gum resin, boswellic acids, emerge as leading non-steroidal anti-inflammatory compounds (drugs) NSAID with broad biological activities and low ulcerogenic index. Preclinical studies established that an alcoholic extract of the gum resin displayed marked anti-inflammatory activity in mice and rats, and also inhibited the formation of leukotrienes in rat peritoneal neutrophils in vitro. Boswellic acids decreased the formation of inflammatory leukotriene B4 (B4 is an outcome of the arachidonic acid metabolism) in rat peritoneal neutrophils in a dose-dependent way with IC50 values ranging from 1.5 to 7 uM. The anti-inflammatory mechanism of action of boswellic acids inhibited the leukotriene synthesis via 5-lopoxygenase, but did not affect the 12-lipoxygenase and cyclooxygenase activity. Additionally, boswellic acids did not impair the peroxidation of arachidonic acid by iron and ascorbate. These results suggest that boswellic acids are specific, non-redox inhibitors of leukotriene synthesis either interacting with 5-lipoxygenase or blocking its translocation.

Safayhi, H. et al (1992) established and prior art by Ammon et al (EP 0 552 657) teaches that six boswellic acids are involved in the inhibition of 5-lipoxygenase, thus potentially blocking synthesis of inflammatory leukotrienes and thus useful in treatment of clinical conditions like inflammatory bowel diseases, arthritis, asthma, psoriasis and chronic form of hepatitis. These six compounds listed by Ammon in order of their biological strength based on IC50-values are as follows: 1. acetyl-11-keto-beta-boswellic acid. 2. Beta-boswellic acid. 3. 11-keto-beta-boswellic acid. 4. Alpha-boswellic acid. 5. Acetyl-beta-boswellic acid and 6. Acetyl-alpha-boswellic acid. Ammon et al (WO 97/07796) also teaches that boswellic acids can be also used as inhibitor of elevated leucocyte elastase or plasmin activity and useful in clinical conditions characterized by the elevated activity of the elastase and/or plasmin. The anti-inflammatory properties of the gum resin is attributed to the presence of “boswellic acids”. Boswellic acids were found to inhibit two pro-inflammatory enzymes, 5-lipoxygenase (which generates inflammatory leukotrienes) and Human Leukocyte Elastase (HLE). HLE is a serine protease which initiates injury to the tissues, which in turn triggers the inflamation. Studies by Safayhi, H. et al (1997) showed that Acetyl-11-keto-β-boswellic acid decreased the activity of human leukocyte elastase HLE) in vitro with an IC₅₀ value of about 15 μM.

Prior art by Lee Yue-Wei et al (U.S. Pat. No. 5,064,823) also teaches that pentacyclic triterpenoid compounds such as alpha boswellic acid and its acetate, beta boswellic acid and its acetate have an inhibitory effect on topoisomerase I and topoisomerase II which according to authors may result in increased cancer cell differentiation. That process may be considered a cancer treatment modality.

An alcoholic extract of the gum resin was examined for anti-carcinogenic properties by Mukherji S. et al (1970). When tested on mice with Ehrlich ascites carcinoma and S-180 tumor, the extract inhibited rumor growth and increased the life span of experimental animals with carcinoma.

### SUMMARY OF THE INVENTION

Despite recognized potential of boswellic acids as NSAIDs and as a promising cancer fighting compounds, there are two major obstacles which stand in way of utilization boswellic acids in the health care: (a) poorly understood relationships between structure/composition of boswellic acids and their biological utility, and (b) lack of the boswellic acids product standardized on the basis of clearly defined structure function claim.

In the present invention, four purified boswellic acids, individually or in mixtures, were discovered to be effective in treating lymphoproliferative conditions and autoimmune diseases in animals, including humans. The four purified boswellic acids were shown, in the present invention, in studies to evaluate the effects against macromolecular biosynthesis and cellular growth of human leukemia HL-60 cells. The four major pentacyclic triterpenic (boswellic) acids present in the acidic extract of Boswellia serrata gum in the present invention are:
β-Boswellic Acid (I)
Acetyl-β-Boswellic Acid (II)
11-keto-β-Boswellic Acid (III)
Acetyl-11-keto-β-Boswellic Acid (IV)

FIGS. 1, 2, and 3 show the inhibitory effects of compounds I-IV on the DNA, RNA and protein synthesis of HL-60 cells, respectively (in FIG. 1-3, lines 1, 2, 3 and 4 refer to the data of compounds I, II, III and IV, respectively). Tables 1 and 2 show the inhibitory effect of a “total organic acids” extract of the exudate of Boswellia serrata on DNA, RNA and protein synthesis or growth in HL-60 cells. Table 3 shows the inhibitory effect of the “total organic acids” extract of the exudate of Boswellia serrata on the incorporation of [³H]thymidine into the DNA of HL-60 cells. The initial rates of incorporation of [³H]-thymidine, [³H]-uridine and [³H]-leucine into trichloroacetic acid (TCA)-insoluble material were utilized to estimate the rates of DNA, RNA, and protein synthesis, respectively, in HL-60 cells. All of no the inhibitory effects of compounds I-IV and the alcoholic extract on DNA. RNA and protein synthesis of HL-60 cells were in a dose-dependent manner. Compounds I, II, III and IV exhibited 50% inhibitory activity on the incorporation of [3H]-thymidine into DNA at concentrations of 3.7, 1.4. 0.9 and 0.6 μM, respectively, the incorporation of [3H]-uridine at concentrations of 7.1, 2.3. 2.2 and 0.5 μM, respectively, and the incorporation of [3H]-leucine into protein at concentrations of 6.3, 5.4, 5.1 and 4.1 μM, respectively, in cultured HL-60 cells incubated for 2 hours.

Comparison of the IC50 values indicated that the order of inhibitory activity for compounds I-IV is IV>III>II>I. This observation is a principle behind the new composition of boswellic acids effective in lymphoproliferative and autoimmune disorders. The discovered relationship between structure and activity of specific boswellic acids in inhibition of DNA, RNA and protein synthesis has not been previously reported. Our research has determined for the first time that (1) 11-keto group of boswellic acids is a principal moiety for the above described biological activity, and (2) 3-0-acetyl group amplifies that activity further resulting in a predictable cytostatic and immunomodulatory effects of boswellic acids.

It has been her determined that compound IV, which induced the most pronounced inhibitory effects on DNA, RNA and protein synthesis in HL-60 cells, had an irreversible inhibitory action on DNA synthesis. In this experiment HL-60 cells were preincubated with compound IV at 2 and 8 μM for 30 min at 37° C., washed with phosphate buffer saline and [3H]-thymidine was added to the culture. At desired times, the reactions were terminated and the rates of DNA synthesis were determined. The results (FIG. 4) showed that the inhibitory effect on DNA synthesis was still dependent upon the concentrations of compound IV and identical to that without washing. This finding suggested that the inhibitory action of compound IV on DNA synthesis was irreversible.

The effect of compound IV on cellular growth of HL-60 cells was tested. As shown in FIG. 8, compound IV depressed the growth of HL-60 cells in a dose-dependent manner. Addition of compound IV at 1, 4, or 16 μM to HL-60 cells and incubation at 37° C. for 4 days inhibited the cellular growth by 54.5, 71.8 or 98.6%. In order to test whether this growth was the result of cell cytotoxicity, the effects of this compound on cell viability were examined after 4 days incubation using the trypan blue exclusion method. The cells viability at concentrations of 0, 1, 4, 16 μM were 97.0, 96.8, 96.5, or 96.7%, respectively.

This experiment showed that compound IV at the concentrations which significantly inhibited cell growth, did not affect cell viability. These results indicated that inhibition of the cell growth is due to the cytostatic rather then cytotoxic effects. The inhibition of cell proliferation can be explained by its interference with biosynthesis of DNA, RNA and protein all of which are required for cell proliferation. These results for the first time establish that composition of boswellic acid enriched with the compound IV can be used as cytostatic and immunomodulatory preparation, due to its profound and well defined effect on myeloid cell metabolism.

Within the scope of the present invention are methods of preventing or treating lymphoproliferative disorders or autoimmune diseases by administering a composition comprising a “total organic acids” extract obtained from Boswellia serrata, administering compound I, II, III or IV individually or administering a mixture comprising two, three or all four of compounds I, II, III and IV in humans or animals in need of such a prevention or treatment. Also within the scope of the present invention are methods of preventing or treating tumors or inflammatory disorders by administering the composition comprising the “total organic acids” extract obtained from Boswellia serrata or administering compound I, II, III or IV individually or administering a mixture comprising two, three or all four of compounds I, II, III and IV in humans or animals in need of such a prevention or treatment. The present invention also includes the composition comprising the “total organic acids” extract obtained from Boswellia serrata, a composition comprising two, three or four of compounds I-IV and two processes of obtaining boswellic acids or of obtaining the composition comprising the “total organic acids” extract obtained from Boswellia serrata.

The lymphoproliferative disorders that can be treated with the methods of using boswellic acids of the present invention include leukemia and lymphoma. Leukemia that can be treated by the methods of the present invention include myeloid leukemia, acute myelogenous leukemia, acute lymphocytic leukemia, acute non-lymphocytic leukemia, chronic lymphocytic leukemia, and hairy cell leukemia. The autoimmune diseases that can be treated with the methods of using boswellic acids of the present invention include, for example, psoriasis, sarcoidosis, systemic lupus erythematosis, Graves' disease, Hashimoto's thyroiditis, silent thyroiditis, Crohn's disease, Goodpasture syndrome, insulin-dependent diabetes mellitus, insulin-resistant diabetes mellitus, myasthenia gravis, Addison's disease, idiopathyic hypoparathyroidism, idiopathic thrombocytopenic purpura, autoimmune hemolytic anemia, rheumatoid arthritis, and scleroderma. The methods of using boswellic acids of the present invention are also effective in treating tumors, including, for example, breast tumors, ovarian tumors, uterine tumor, lung tumors, liver tumors, renal tumors, prostatic tumors, pancreatic tumors, tumors of the gastrointestinal tract e.g. colorectal tumors, brain tumors, and head and neck tumors,

The following tables present data concerning the biological effects of an alcoholic extract of the exudate of Boswellia serrata. Table 1 below presents data on the effects of the alcoholic extract of the exudate of Boswellia serrata on the DNA synthesis, RNA synthesis and protein synthesis in HL-60 cells in culture.

TABLE 1

| BSE | DNA synthesis | | RNA syntheses | | Protein synthesis | |
|---|---|---|---|---|---|---|
| added | % | % | % | % | % | % |
| (μm) | Control | Inhibition | Control | Inhibition | Control | Inhibition |
| 0 | 100 | 0 | 100 | 0 | 100 | 0 |
| 0.75 | 80 | 20 | 91 | 9 | 70 | 30 |
| 1.5 | 45 | 55 | 64 | 36 | 52 | 48 |
| 3.0 | 35 | 65 | 62 | 38 | 26 | 74 |
| 6.0 | 23 | 77 | 20 | 80 | 12 | 88 |
| 12.0 | 19 | 81 | 10 | 90 | 9 | 91 |
| 25.0 | 18 | 82 | 8 | 92 | 8 | 92 |

Various concentrations of the Boswellia serrata extract, as indicated above, were added to 1 mL of HL-60 cells suspended in RPMI medium. [³H]thymidine (50 μCi/μmol: 3 mL), [³H]uridine (55 μCi/μmol; 5 μL), [³H]leucine (200 μCi/μmol: 10 μL), were added to the cell suspension and incubated at 37° C. for 120 min. Reactions were terminated by addition of 3 mL of cold PBS, and the rates of DNA. RNA, and protein synthesis were determined.

Table 2 below presents data on the effect of the alcoholic extract of the exudate of Boswellia serrata on the growth of HL-60 cells in culture. The alcoholic extract of the exudate of Boswellia serrata inhibited the growth of HL-60 cells in a concentration dependent fashion.

TABLE 2

| Incubation | | | | |
|---|---|---|---|---|
| time | Concentration of BSE (μM) | | | |
| (hours) | 0 | 4 | 12 | 50 |
| 0 | 25 ± 2.3 | 25 ± 2.3 | 25 ± 2.3 | 25 ± 2.3 |
| 24 | 45 ± 2.1 | 40 ± 4.2 | 39 ± 3.7 | 30 ± 4.0 |
| | | (25%) | (30%) | (75%) |
| 48 | 71 ± 1.5 | 66 ± 4.7 | 57 ± 3.5 | 27 ± 2.0 |
| | | (11%) | (30%) | (97%) |
| 72 | 102 ± 2.1 | 95 ± 2.9 | 72 ± 7.8 | 25 ± 1.2 |
| | | (9%) | (40%) | (100%) |
| 96 | 166 ± 16.6 | 159 ± 11 | 102 ± 2.6 | 31 ± 2.2 |
| | | (5%) | (45%) | (96%) |

Various concentrations of BSE, as indicated above, were added to the HL-60 cell cultures. These cultures were counted daily using a hemacytometer under a microscope with 10× magnification every 24 hours. Data are expressed as the mean=SE calculated from triplicate studies. Data in parentheses are the percent inhibition of cell growth.

Other than the inhibitory effects on the synthesis of RNA and protein in HL-60 cells grown in culture, the present invention demonstrated that boswellic acids have an inhibitory effect on DNA synthesis in HL-60 cells. Table 3 below shows that the alcoholic extract of the exudate of Boswellia serrata can inhibit DNA synthesis in HL-60 cells as demonstrated by an inhibition of the incorporation of ³H-labeled thymidine into the DNA of HL-60 cells. Similar to the results in Table 2. Table 3 demonstrates that the inhibitor, effect of the alcoholic extract of the exudate of Boswellia serrata on DNA synthesis in HL-60 cells exhibited a concentration dependent response.

TABLE 3

| Incubation | Concentration of BSE (μM) | | | |
|---|---|---|---|---|
| time | 0 | 4 | 12 | 50 |
| (min) | (cpm/5 × 10⁵ cells) | | | |
| 0 | 279 ± 76 | 352 ± 114 | 312 ± 54 | 225 ± 15 |
| 120 | 11112 ± 1897 | 4039 ± 737 | 2794 ± 306 | 1893 ± 505 |
| | | (69%) | (77%) | (86%) |

[³H]Thymidine (3 μL; 50 μCi/μmol), vehicle or various concentrations of BSE in vehicle were added to 1 mL of HL-60 cells (5×10⁵ cells/mL) in culture, and the cultures were incubated at 37° C. for 120 min. Data are expressed as the mean±SE calculated from triplicate studies. Data in parentheses are the percent inhibition of [³H]thymidine incorporation into the DNA of HL-60 cells.

### BRIEF DESCRIPTION OF THE DRAWINGS

FIG. 1 depicts the effects of compounds I-IV on the DNA synthesis in HL-60 cells.

FIG. 2 depicts the effects of compounds I-IV on the RNA synthesis in HL-60 cells.

FIG. 3 depicts the effects of compounds I-IV on the protein synthesis in HL-60 cells.

FIG. 4 shows the inhibitory effects of compound IV on the DNA synthesis in HL-60 cells.

FIGS. 5, 6 and 7 show the β-boswellic acids contents in 6 commercial samples of Boswellia serrata extract.

FIG. 8 shows the inhibitory effect of compound IV on the growth of HL-60 cells.

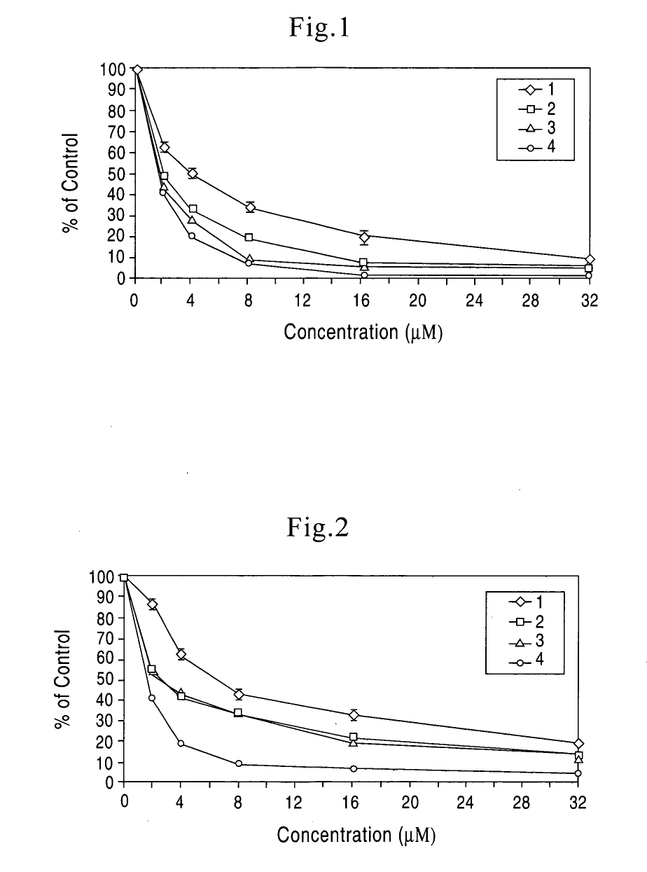

### DETAILED DESCRIPTION OF THE INVENTION

Based on our experimental data on relationship between structure and function of the four boswellic acids of invention, a novel manufacturing and standardization process for boswellic acids have been developed. The new standardization process resulted in changes in the nomenclature of the boswellic acids preparation. The new nomenclature included the following changes.

The phrase “total organic acids” from Boswellia serrata refers to an organic acid fraction of an extract of Boswellia serrata or Boswellia serrata gum. The “total organic acids” from Boswellia serrata constitute approximately 65-70%. by weight, of the total alcoholic extract of Boswellia serrata. In the methods of treatment of the present invention, the daily effective dose, for a 70 kg subject to be treated, is 1-5000 mg “total organic acids” from Boswellia serrata. 2 to 4 times a day. The preferred daily effective dose is 10-500 mg “total organic acids”, 2 to 4 times a day. The more preferred daily effective dose is 100-400 mg “total organic acids”, 2 to 4 times a day. The most preferred daily effective dose is 200 mg “total organic acids”, 3 times a day. For humans or animals of a body weight other than 70 kg, the above doses can be adjusted accordingly based on the body weight or the body surface area based on methods known in the art.

The term “pure boswellic acids” indicates the four major boswellic acids in each dosage form. The “pure boswellic acids” can contain two, three or all four of the four major boswellic acids, i.e. β-boswellic acid (1), acetyl-β-boswellic acid (II), 11-keto-β-boswellic acid (I), and acetyl-11-keto-β-boswellic acid (IV). The “pure boswellic acids” constitute approximately 25% of the “total organic acids”. In the methods of treatment of the present invention, the daily effective dose, for a 70 kg subject to be treated, is 0.25-1250 mg “pure boswellic acids”, 2 to 4 times a day. The preferred daily effective dose is 2.5-125 mg “pure boswellic acids”, 2 to 4 times a day. The more preferred daily effective dose is 25-100 mg “pure boswellic acids”, 2 to 4 times a day. The most preferred daily effective dose is 50 mg “pure boswellic acids”, 3 times a day. For humans or animals of a body weight other than 70 kg, the above doses can be adjusted accordingly based on the body weight or the body surface area based on methods known in the art.

The total organic acids extract from Boswellia serrata can be administered by topical, inhalational, parenteral or oral routes, or by nasal spray or suppositories. Similarly, pure boswellic acids, individual boswellic acids, or mixtures thereof, can be administered by topical, inhalational, parental or oral routes, or by nasal spray or suppositories.

Although there are other components in the Boswellia serrata gum (e.g. alpha and gamma-Boswellic acids), the four major pentacyclic triterpenic (boswellic) acids present in the acidic extract of Boswellia serrata gum of the invention used for standardization are:
β-Boswellic Acid (I)
Acetyl-β-Boswellic Acid (II)
11-keto-β-Boswellic Acid (III)
Acetyl-11-keto-β-Boswellic Acid (IV)

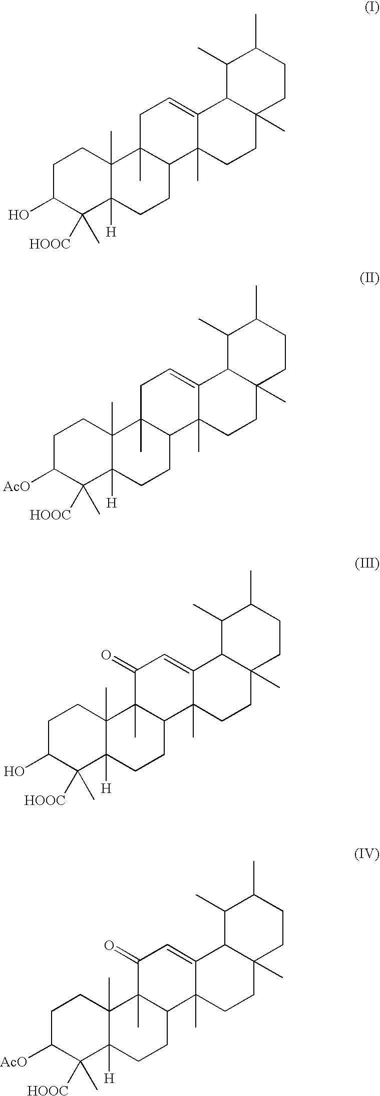

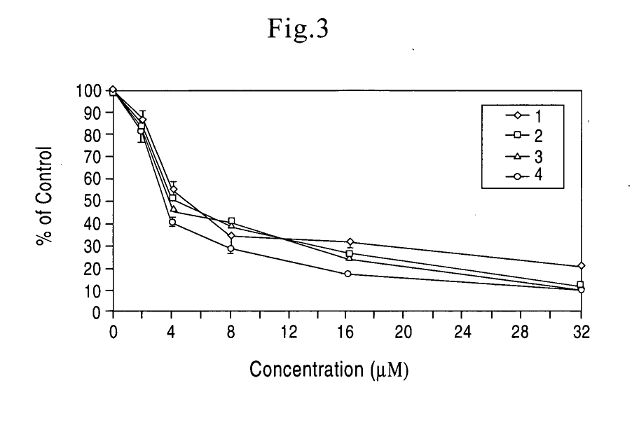

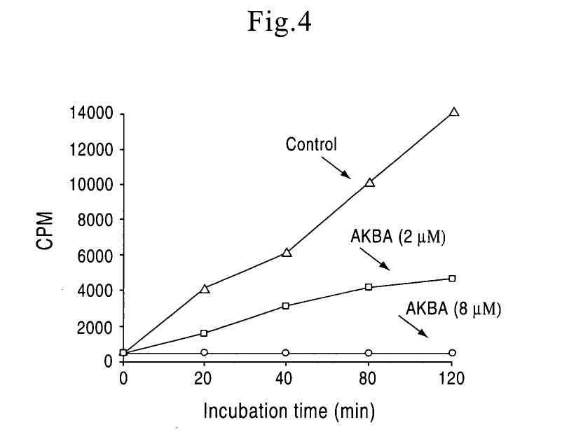

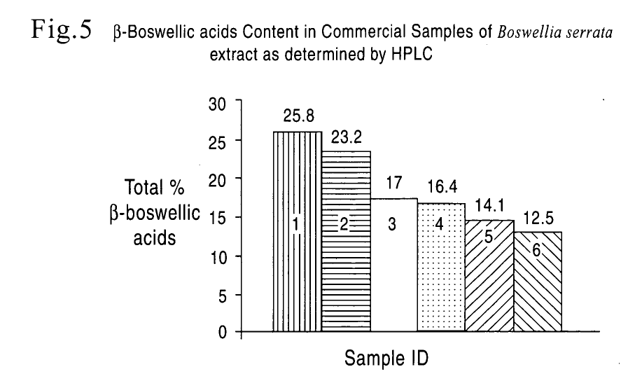

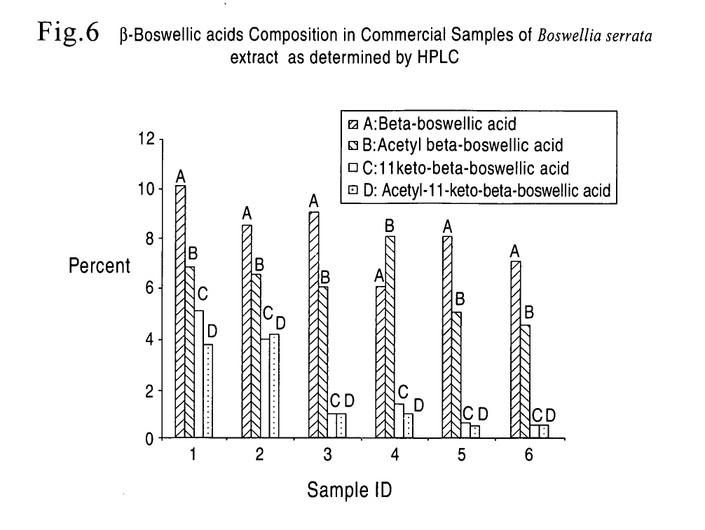

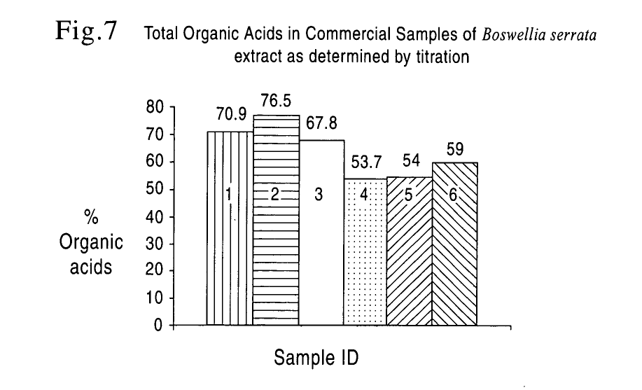

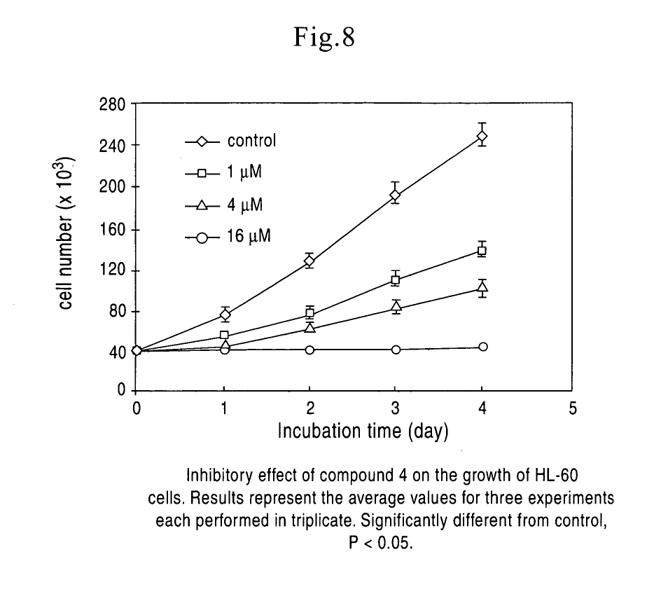

Commercial samples of Boswellia serrata extracts vary greatly in their contents of boswellic acids, which limits, as previously mentioned a reliable use of boswellic acids in medical and veterinary applications. The analytical results for six commercial samples are indicated in FIG. 3, FIG. 6 and FIG. 7, in terms of content of boswellic acids, their composition, and total organic acids content respectively. In many commercial samples, the most active β-Boswellic acids are available in negligible quantities only. The total organic acids content in these samples as determined by titration is indicated in FIG. 7.

The above analytical results make it evident that (a) there is need for accurately standardized boswellic acid product by the HPLC method and (b) that the active components in Boswellia serrata extract cannot be accurately predicted based on titrimetric method analysis. It is equally interesting to note that while the titrimetric method gives more than 50% by weight of organic acids, several of the commercially available products contain only negligible amounts of the two key boswellic acids, namely 11-keto-beta- and acetyl-11-keto-beta-boswellic acids (FIG. 6).

Method of Extraction of Boswellic Acids

By applying a prior art extraction method on a typical sample of Boswellia serrata, a composition was obtained containing the four boswellic acids, compounds I-IV, at concentrations shown below:

| Component | % by weight |
|---|---|
| I. β-Boswellic Acid | 10.1 |
| II. Acetyl-β-Boswellic Acid | 6.8 |
| III. 11-keto-β-Boswellic Acid | 5.1 |
| IV. Acetyl-11-keto-β-Boswellic Acid | 3.8 |
| Total | 25.8 |

The “total organic acids” value of this preparation by titration method was: 70.9% by weight.

The present invention includes a first new process of extraction to obtain boswellic acids to ascertain a minimum yield of total boswellic acids by HPLC of minimum 38 weight %, with compound IV of not less than 4 weight %. compound III of not less than 5 weight %, compound II of not less than 10 weighty % and compound I of not less than 14 weight %. The yield of boswellic acids obtainable by the first new process of the present invention is much higher than the prior art process of extraction. Flow chart of old process versus the first new extraction and manufacturing process is shown below.

PROCESS COMPARISON

| OLD PROCESS | NEW PROCESS |
|---|---|
| 1. Boswellia serrata | 1. Boswellia serrata |
| 2. Extract with hot isopropyl alcohol | 2. Extract with hot C₁-C₆ alcohol, |
| | e.g. isopropyl alcohol, butanol |
| 3. Concentrate the isopropyl alcohol | 3. Strip off the alcohol extract |
| extract to 50% | completely |
| 4. Treat with KOH to pH 9.5 at | 4. Treat with an alkaline substance, |
| 60° C. | e.g. alkali such as KOH or |
| | NaOH, to pH > 9.5 at room |
| | temperature |
| 5. Remove isopropyl alcohol and | 5. Wash with an organic solvent, |
| wash with ether | such as an ester or ketone |
| | solvent |
| 6. Treat aqueous layer with | 6. Treat aqueous layer with |
| hydrochloric acid to pH 4 | hydrochloric acid to pH 4 |
| 7. Obtain precipitate | 7. Obtain precipitate |
| 8. Wash precipitate with water | 8. Wash precipitate with water |
| 9. Dry the precipitate | 9. Dry the precipitate at <50° C. |

In the first new process of extraction to obtain boswellic acids, an example of the organic solvent used in step 5 is ethyl acetate. As needed, modifications, obvious to one skilled in the art, of the new process of extraction to obtain boswellic acids can be done. The modified new process of extraction is also within the scope of the present invention.

Example of manufacturing process of boswellic acid of invention

Process Data Sheet For The Manufacture Of Boswellin 100 kg

1. Charge the extractor with Boswellia serrata gum 555 kg.
2. Charge isopropyl alcohol to the soaking level (1100 L-false bottom capacity).
3. Pass steam into the jacket and maintain the temperature at 68-70 deg. C. in the core body of the reactor.
4. Drain the extract into a reactor and concentrate at 70 deg. C. to strip off isopropyl alcohol completely.
5. Charge isopropyl alcohol to the soaking level 550 L and repeat the step 3 to 4
6. Repeat step 5
7. Charge 560 L of 5 weighted aqueous KOH, then stir at room temperature for 3 hours.
8. Wash with ethyl acetate 830 L.
9. Drain the ethyl acetate layer and collect aqueous layer.
10. Repeat step 8 and 9 two times with 550 L ethylacetate and collect the aqueous layer.
11. Charge the aqueous layer (from steps 9 and 10) into a reactor.
12. Add slowly 6 N HCl to pH 34 (˜30 L) while stirring at room temperature.
13. Forms a precipitate.
14. Add 1000 L of water and let it stand at room temperature for 8 hours (or less depending on the observation).
15. Collect the precipitate (by draining into a nutsch and scooping), wash with water.
16. Check for Boswellin in aqueous portion, if absent discard.
17. Dry the prcipitate not above 50 deg. C.
18. Yield expected ˜100 kg (assay by HPLC 3840%).
   Assay by HPLC for Beta Boswellic Acids
Mobile phase:
Mobile phase A: 1000 ml of Acetonitrile with 0.05 ml (1 drop) of glacial acetic acid, filter and degas.
Mobile phase B: Mix water and acetonitrile in the ratio 150:850 with 0.05 ml (1 drop) of glacial acetic acid filter and degas.

Use gradient program

| Time | A concentration | B concentration |
|---|---|---|
| 0 min | 90% | 10% |
| 15 min | 20% | 80% |
| 20 min | 0% | 100% |
| 25 min | 50% | 50% |
| 30 min | 100% | 0% |
| 30 min | stop | |

Sample Preparation:
Weigh accurately about 200 mg of the sample and transfer into a 50 ml volumetric flask. Add 25 ml of methanol to dissolve the sample, and sonicate for 3 minutes, dilute to volume, mix.
Standard Preparation:
1. Beta-boswellic acid: weigh accurately about 25 mg of the standard and transfer into a 10 ml volumetric flask. Add 5 ml of methanol to dissolve the sample, sonicate for 3 minutes, dilute to volume, mix.
2. Acetyl-beta-boswellic acid: weigh accurately about 500 mg of standard and transfer into a 10 ml volumetric flask. Add 5 ml of methanol to dissolve the sample, sonicate for 3 minutes, dilute to volume, mix.
3. 11-Keto-beta-boswellic acid; weigh accurately about 25 mg of the standard and transfer into a 25 ml volumeric flask. Add 15 ml of methanol to dissolve the sample, sonicate for 3 minutes, dilute to volume, mix.
4 Acetyl-11-keto-beta-boswellic acid: weigh accurately about 25 mg of the standard and transfer into a 25 ml volumetric flask. Add 15 ml of methanol to dissolve the sample, sonicate for 3 minutes, dilute to volume, mix,
   Alteratively, weigh accurately about 25 mg of the standard (which contains known concentration of beta-boswellic acid) into 25 ml volumetric flask. Add 15 ml of methanol to dissolve the sample, sonicate for 3 minutes, dilute to volume, mix.
   Chromatographic System:
   The liquid chromatograph is equipped with 210 nm and 256 nm UV detector and a 250×4.6 mm column that contains the packing C18 or ODS (Sigma/Aldrich column is used). The flow rate is 1.0 ml per min. The relative standard deviation for replicate injection of Standard preparation should not be more than 2%.
   Procedure:
   Separately inject equal volume (20 ul) of the standard preparations and sample preparation into the chromatograph, record the responses for the peak of beta-boswellic acid and acetyl-beta-boswellic acid at 210 nm and for the peaks of 11-keto-beta-boswellic acid and acetyl-11-ketoboswellic acid at 245 nm and calculate the percentage by weight of each boswellic acids as follows:

The following are the retention times of the four beta Boswellic acids:

| | |
|---|---|
| 1. Beta-boswellic acid | 17.4 min |
| 2. 3-acetyl beta-boswellic acid | 26.0 min |
| 3. 11-keto-beta-boswellic acid | 7.2 min |
| 4. 3-acetyl-11-keto-beta-boswellic acid | 10.4 min |

Area of Sample × Standard concentration in mg/ml × Purity of the standard
Area of Standard × Sample concentration in mg/ml

Results of HPLC Assay of Pentacyclic Triterpinic Acids

| | | RD/BS/21 | New Trial |
|---|---|---|---|
| | Old Plant | New R&D | Plant Batch |
| Description | Batch | Batch (1 kg) | (100 kg) |
| Beta-Boswellic acid | 10.3 wt % | 15 wt % | 14 wt % |
| Acetyl-beta-boswellic acid | 7.1 wt % | 11 wt % | 13.5 wt % |
| 11-keto-boswellic acid | 3.3 wt % | 6.5 wt % | 6.5 wt % |
| Acetyl-keto-beta-boswellic acid | 3.4 wt % | 7.6 wt % | 7.5 wt % |
| TOTAL % | 24.1 wt % | 40.1 wt % | 41.5 wt % |

Wherein “Old” means the old process and “New” means the new process.

The “total organic acids” extract of the present invention can be obtained by a process comprising the following steps:

(1) providing a Boswellia serrata component;

(2) extracting the component with a C₁-C₆ alcohol. e.g. isopropyl alcohol, to obtain an alcohol extract;

(3) remove the C₁-C₆ alcohol from the alcohol extract to obtain a liquid;

(4) treat the liquid with an alkaline substance, such as an alkali, e.g. KOH, to obtain an alkaline liquid;

(5) wash the alkaline liquid with an organic solvent, e.g. ethyl acetate;

(6) remove the organic solvent to obtain an aqueous liquid; and thereafter

(7) treat the aqueous liquid with an acid. e.g. hydrochloric acid, to form the “total organic acids” extract as a precipitate.

Preferably, the Boswellia serrata component used is Boswellia serrata gum. The component in step (2) is preferably treated with hot isopropyl alcohol at a temperature of about 50-80° C. about 60-75° C. about 68-72° C. or about 70° C. The treatment with KOH in step (4) preferably is carried out at pH>9.5. Step (7) is preferably conducted by treating the aqueous liquid with hydrochloric acid at about pH 3 to 4 to obtain a precipitate, which optionally can be washed with water and dried at a temperature less than about 50° C.

From the “total organic acids” extract obtained by the new process of the present invention, individual pure oswellic acids. i.e. compounds I, II, III or IV, can be obtained by chromatographic methods known in the prior art. The pure compound I, II, III and IV can also be obtained by synthetic processes known in the art. The individual pure oswellic acid can be mixed in any ratio to obtain desired mixtures.

The present invention includes compositions comprising the “total organic acids” extract obtained by the new process of the invention, any one of pure compound I, II, III or IV, or mixtures of two, three or all of compounds I-IV, mixed with a physiologically acceptable carrier or excipient.

The compositions of the present invention can comprise compound I:compound II:compound III:compound IV in any proportions. Preferably, the compositions comprise compound I:compound II:compound III:compound IV of 10-20:5-25:1-15:1-1-20 (or 15-20:5-25:1-15:1-20). More preferably, the compositions comprise compound I:compound II:compound III:compound IV of 12-17:-18:3-10:2-15. Much preferred compositions of the present invention comprise compound I:compound II:compound III:compound IV of 14-16:8-17:4-9:3-10. Most preferred compositions of the present invention comprise compound I:compound II:compound III:compound IV of 15:10-15:5-8:4-8.

Another aspect of the present invention is a composition consisting essentially of, based on the total weight of the composition, β-boswellic acid of at least 12% by weight acetyl-β-boswellic acid of at least 5% by weight, 11-keto-β-boswellic acid of at least 1% by weight and acetyl-11-keto-β-boswellic acid of at least 1% by weight. This composition can contain other boswellic acids, e.g. 3a-hydroxy-urs-9,12-diene-24-oic acid or 2a,3a-dihydroxy-urs-12-ene-24-oic acid, each of which at a content of less than 1% by weight, based on the total weight of the composition. Preferably, the composition consists essentially of, based on the total weight of the composition, β-boswellic acid of at least 14% by weight, acetyl-β-boswellic acid of at least 5% by weight, 11-keto-β-boswellic acid of at least 5% by weight and acetyl-11-keto-β-boswellic acid of at least 5% by weight. Also preferably, the composition consists essentially of, based on the total weight of the composition, β-boswellic acid of 12 to 35% by weight, acetyl-β-boswellic acid of 5 to 35% by weight, 11-keto-β-boswellic acid of 5 to 45% by weight and acetyl-11-keto-β-boswellic acid of 5 to 45% by weight. The composition, also preferably, consists essentially of, based on the total weight of the composition, β-boswellic acid of 12 to 30% by weight, acetyl-β-boswellic acid of 10 to 25% by weight, 11-keto-β-boswellic acid of 5 to 35% by weight and acetyl-11-keto-β-boswellic acid of 5 to 35% by weight. More preferably, the composition consists essentially of, based on the total weight of the composition, β-boswellic acid of 14 to 30% by weight, acetyl-β-boswellic acid of 10 to 20% by weight, 11-keto-β-boswellic acid of 5 to 25% by weight and acetyl-11-keto-β-boswellic acid of 5 to 25% by weight. Also more preferably, the composition consists essentially of, based on the total weight of the composition, β-boswellic acid of 14 to 35% by weight, acetyl-β-boswellic acid of 10 to 20% by weight, 11-keto-β-boswellic acid of 5 to 25% by weight and acetyl-11-keto-β-boswellic acid of 5 to 20% by weight. Also more preferably, the composition consists essentially of, based on the total weight of the composition, β-boswellic acid of 14 to 35% by weight, acetyl-β-boswellic acid of 10 to 20% by weight, 11-keto-β-boswellic acid of 5 to 20% by weight and acetyl-11-keto-β-boswellic acid of 5 to 25% by weight.

Another aspect of the present invention is a composition comprising three boswellic acids selected from the group consisting of β-boswellic acid, acetyl-β-boswellic acid, 11-keto-β-boswellic acid and acetyl-11-keto-β-boswellic acid, wherein, based on the total weight of the composition, the amount of β-boswellic acid is at least 5% by weight, the amount of acetyl-β-boswellic acid of is least 5% by weight, the amount of 11-keto-β-boswellic acid is at least 5% by weight, and the amount of acetyl-11-keto-β-boswellic acid is at least 5% by weight. Preferably, in the composition, the amount of β-boswellic acid is 14 to 65% by weight, the amount of acetyl-β-boswellic acid is 5 to 65% by weight, the amount of 11-keto-β-boswellic acid is 5 to 60% by weight, and the amount of acetyl-11-keto-β-boswellic acid is 5 to 60% by weight. Also preferably, in the composition, the amount of β-boswellic acid is 14 to 55% by weight, the amount of acetyl-β-boswellic acid is 10 to 55% by weight, the amount of 11-keto-β-boswellic acid is 5 to 50% by weight, and the amount of acetyl-11-keto-β-boswellic acid is 5 to 50% by weight. Also preferably, in the composition, the amount of β-boswellic acid is 14 to 35% by weight, the amount of acetyl-β-boswellic acid is 10 to 35% by weight, the amount of 11-β-boswellic acid is 5 to 40% by weight, and the amount of acetyl-11-keto-β-boswellic acid is 5 to 40% by weight. Also preferably, in the composition, the β-boswellic acid, acetyl-β-boswellic acid, 11-keto-β-boswellic acid and acetyl-11-keto-β-boswellic acid are derived from any natal source. Also preferably, in the composition, two of the three boswellic acids are 11-keto-β-boswellic acid and acetyl-11-keto-β-boswellic acid.

Another aspect of the present invention is a composition comprising two boswellic acids selected from the group consisting of β-boswellic acid, acetyl-β-boswellic acid, 11-keto-β-boswellic acid and acetyl-11-keto-β-boswellic acid, wherein based on the total weight of the composition, the amount of β-boswellic acid is at least 5% by weight, the amount of acetyl-β-boswellic acid is least 5% by weight the amount of 11-keto-β-boswellic acid is at least 5% by weight, and the amount of acetyl-11-keto-β-boswellic acid is at least 5% by weight. Preferably, in the composition, the amount of β-boswellic acid is 5 to 95% by weight, the amount of acetyl-β-boswellic acid is 5 to 95% by weight, the amount of 11-keto-β-boswellic acid is 5 to 95% by weight, and the amount of acetyl-11-keto-β-boswellic acid is 5 to 95% by weight. Preferably, in the composition, the amount of β-boswellic acid is 30 to 70% by weight the amount of acetyl-β-boswellic acid is 30 to 70% by weight, the amount of 11-keto-β-boswellic acid is 30 to 70% by weight, and the amount of acetyl-11-keto-β-boswellic acid is 30 to 70% by weight. Also preferably, in the composition, the amount of β-boswellic acid is 40 to 60% by weight, the amount of acetyl-β-boswellic acid is 40 to 60% by weight, the amount of 11-keto-β-boswellic acid is 40 to 60% by weight, and the amount of acetyl-11-keto-β-boswellic acid is 40 to 60% by weight. Also preferably, in the composition, the two boswellic acids are 11-keto-β-boswellic acid and acetyl-11-keto-β-boswellic acid.

Within the scope of the present invention is a composition comprising boswellic acids, wherein the boswellic acids consist of three substances selected from the group consisting of β-boswellic acid, acetyl-β-boswellic acid, 11-keto-β-boswellic acid and acetyl-11-keto-β-boswellic acid, wherein, based on the total weight of the composition, the amount of β-boswellic acid is at least 5% by weight, the amount of acetyl-β-boswellic acid is least 5% by weight, the amount of 11-keto-β-boswellic acid is at least 5% by weight, and the amount of acetyl-11-keto-β-boswellic acid is at least 5% by weight. Preferably, in the composition, the amount of β-boswellic acid is 5 to 65% by weight, the amount of acetyl-β-boswellic acid is 5 to 65% by weight, the amount of 11-keto-β-boswellic acid is 5 to 65% by weight, and the amount of acetyl-11-keto-β-boswellic acid is 5 to 65% by weight. Also preferably, in the composition, the amount of β-boswellic acid is 15 to 55% by weight the amount of acetyl-β-boswellic acid is 15 to 55% by weight, the amount of 11-keto-β-boswellic acid is 15 to 55% by weight, and the amount of acetyl-11-keto-β-boswellic acid is 15 to 55% by weight. Also preferably, in the composition, the amount of β-boswellic acid is 20 to 40% by weight, the amount of acetyl-β-boswellic acid is 20 to 40% by weight, the amount of 11-keto-β-boswellic acid is 20 to 40% by weight and the amount of acetyl-11-keto-β-boswellic acid is 20 to 40% by weight. Also preferably, in the composition, two of the three substances are 11-keto-β-boswellic acid and acetyl-11-keto-β-boswellic acid.

Another aspect of the present invention is a composition comprising boswellic acids, wherein the boswellic acids consist of two substances selected from the group consisting of β-boswellic acid, acetyl-β-boswellic acid, 11-keto-β-boswellic acid and acetyl-11-keto-β-boswellic acid, wherein, based on the total weight of the boswellic acids, the amount of β-boswellic acid is at least 5% by weight, the amount of acetyl-5-boswellic acid of is least 5% by weight, the amount of 11-keto-β-boswellic acid is at least 5% by weight, and the amount of acetyl-11-keto-β-boswellic acid is at least 5% by weight. Preferably, in the composition, the amount of β-boswellic acid is 10 to 90% by weight, the amount of acetyl-β-boswellic acid is 10 to 90% by weight, the amount of 11-keto-β-boswellic acid is 10 to 90% by weight, and the amount of acetyl-11-keto-β-boswellic acid is 10 to 90% by weight. Also preferably, in the composition, the amount of β-boswellic acid is 20 to 80% by weight, the amount of acetyl-β-boswellic acid is 20 to 80% by weight, the amount of 11-keto-β-boswellic acid is 20 to 80% by weight, and the amount of acetyl-11-keto-β-boswellic acid is 20 to 80% by weight. Also preferably, in the composition, the amount of β-boswellic acid is 30 to 70% by weight, the amount of acetyl-β-boswellic acid is 30 to 70% by weight, the amount of 11-keto-β-boswellic acid is 30 to 70% by weight, and the amount of acetyl-11-keto-β-boswellic acid is 30 to 70% by weight. Also preferably, in the composition, the amount of boswellic acid is 40 to 60% by weight the amount of acetyl-β-boswellic acid is 40 to 60% by weight, the amount of 11-keto-β-boswellic acid is 40 to 60% by weight, and the amount of acetyl-11-keto-β-boswellic acid is 40 to 60% by weight. Also preferably, in the composition, the two substances are 11-keto-β-boswellic acid and acetyl-11-keto-β-boswellic acid.

Another embodiment of the present invention is a method for inhibition of DNA, RNA and/or protein synthesis in a human or animal in need of the inhibition, wherein the method comprises a step of administering a DNA, RNA anchor protein synthesis inhibition effective amount of a composition to said human or animal, wherein the composition comprises β-boswellic acid, acetyl-β-boswellic acid, 11-keto-β-boswellic acid and acetyl-11-keto-β-boswellic acid. Preferably, the composition comprises β-boswellic acid of at least 12% by weight, acetyl-β-boswellic acid of at least 5% by weight, 11-keto-β-boswellic acid of at least 1% by weight and acetyl-11-keto-β-boswellic acid of at least 1% by weight. More preferably, the composition comprises β-boswellic acid of 12 to 35% by weight, acetyl-β-boswellic acid of 5 to 35% by weight, 11-keto-β-boswellic acid of 5 to 45% by weight and acetyl-11-keto-β-boswellic acid of 5 to 45% by weight.

Another embodiment of the present invention is a method for irreversible inhibition of DNA synthesis in a human or animal in need of the inhibition, comprising a step of administering an irreversible DNA inhibition effective amount of a composition to said human or animal, wherein the composition comprises β-boswellic acid, acetyl-β-boswellic acid, 11-keto-β-boswellic acid and acetyl-11-keto-β-boswellic acid. Preferably, for used in the method, the composition comprises β-boswellic acid of at least 12% by weight, acetyl-β-boswellic acid of at least 5% by weight, 11-keto-β-boswellic acid of at least 1% by weight and acetyl-11-keto-β-boswellic acid of at least 1% by weight. For used in the method, the composition more preferably comprises β-boswellic acid of 12 to 35% by weight, acetyl-boswellic acid of 5 to 35% by weight, 11-keto-β-boswellic acid of 5 to 45% by weight and acetyl-11-keto-β-boswellic acid of 5 to 45% by weight,

Within the scope of the present invention is a method for the prevention or treatment of a lymphoproliferative disease in a human or animal in need of the prevention or treatment, wherein the method comprises a step of administering a lymphoproliferative disease prevention or treatment effective amount of a composition to said human or animal, wherein the composition comprises β-boswellic acid, acetyl-β-boswellic acid, 11-keto-β-boswellic acid and acetyl-11-keto-β-boswellic acid. Preferably, for used in the method, the composition comprises β-boswellic acid of at least 12% by weight, acetyl-β-boswellic acid of at least 5% by weight, 11-keto-β-boswellic acid of at least 1% by weight and acetyl-11-keto-β-boswellic acid of at least 1% by weight. More preferably, for used in the method, the composition comprises β-boswellic acid of 12 to 35% by weight, acetyl-β-boswellic acid of 5 to 35% by weight, 11-keto-β-boswellic acid of 5 to 45% by weight and acetyl-11-keto-β-boswellic acid of 5 to 45% by weight.

Another aspect of the present invention is a method for the prevention or treatment of an autoimmune disease in a human or animal in need of the prevention or treatment, wherein the method comprises a step of administering an autoimmune disease prevention or treatment effective amount of a composition to said human or animal, wherein the composition comprises β-boswellic acid, acetyl-β-boswellic acid, 11-keto-β-boswellic acid and acetyl-11-keto-β-boswellic acid. Preferably, for used in the method, the composition comprises β-boswellic acid of at least 12% by weight, acetyl-β-boswellic acid of at least 5% by weight, 11-keto-β-boswellic acid of at least 1% by weight and acetyl-11-keto-β-boswellic acid of at least 1% by weight. More preferably, for used in the method, the composition comprises β-boswellic acid of 12 to 35% by weight, acetyl-β-boswellic acid of 5 to 35% by weight, 11-keto-β-boswellic acid of 5 to 45% by weight and acetyl-11-keto-β-boswellic acid of 5 to 45% by weight.

Another aspect of the present invention is a method of inhibiting the synthesis of DNA, RNA and/or protein in a human or animal in need of the inhibition, comprising administering a DNA, RNA and/or protein synthesis inhibition effective amount of β-boswellic acid, acetyl-β-boswellic acid 11-keto-β-boswellic acid or acetyl-11-keto-β-boswellic acid.

Another aspect of the present invention is a method for irreversibly inhibiting the synthesis of DNA in a human or animal in need or the inhibition, comprising administering a DNA synthesis reversible inhibition effective amount of β-boswellic acid, acetyl-β-boswellic acid, 11-keto-β-boswellic acid or acetyl-11-keto-β-boswellic acid.

Another aspect of the present invention is a method for preventing or treating a lymphoproliferative disease in a human or animal in need of the prevention or treatment comprising administering a lymphoproliferative disease preventing or treating effective amount of β-boswellic acid, acetyl-β-boswellic acid, 11-keto-β-boswellic acid or acetyl-11-keto-β-boswellic acid.

Another aspect of the present invention is a method for preventing or treating an autoimmune disease in a human or animal in need of the prevention or treatment, comprising administering an autoimmune disease preventing or treating effective amount of β-boswellic acid acetyl-3-boswellic acid, 11-keto-β-boswellic acid or acetyl-11-keto-β-boswellic acid.

Also within the scope of the present invention are methods of using the compositions or boswellic acid(s), individually or mixtures thereof, of the present invention to make a medication for inhibiting the synthesis of DNA, RNA and/or protein, for irreversibly inhibiting the synthesis of DNA, for preventing or treating a lymphoproliferative or autoimmune disease.

Also preferably, in the compositions of the present invention, the β-boswellic acid acetyl-β-boswellic acid 11-keto-β-boswellic acid and acetyl-11-keto-β-boswellic acid are derived from any natural source.

Within the scope of the present invention is a second new extraction process to obtain boswellic acids from Boswellia serrata. The second new extraction process of obtaining boswellic acids comprises the following steps:

(a) providing a Boswellia serrata component;

(b) extracting said Boswellia serrata component with carbon dioxide to obtain a fluid extract; and

(c) removing carbon dioxide from the fluid extract to obtain the boswellic acids.

In the second new extraction process, the Boswellia serrata component preferably is a gum or degummed resin from Boswellia serrata. The extracting step in the second new extraction process can be performed with subcritical extraction or supercritical extraction using liquid carbon dioxide. After the removal of carbon dioxide from the fluid extract, the so obtained boswellic acids can be, if necessary, subjected to further separation or purification, such as chromatography or selective precipitation in appropriate organic solvents.

Carbon dioxide may be used as an extracting solvent in either of two forms—subcritical and supercritical. Carbon dioxide has a critical temperature of 31.2° C. and a critical pressure of 73.8 bars (1070 psi). The subcritical extraction is performed in the liquid state at a pressure in the range of 300 to 700 psi (20 to 48 bars) and a temperature or temperatures ranging from 0° to 31° C. The supercritical extraction is performed in the fluid gas state at a temperature or temperatures above the critical temperature (31.2° C. or 89° F.) and a pressure in the range of 2000 to 4000 psi (138 to 275 bars). The second new extraction process using supercritical extraction gives a higher yield in a shorter time.

For subcritical extractions, high pressure batch or continuous extraction systems may be used. For supercritical extractions, suitable equipment includes packed or plate columns, towers featuring perforated plates or baffle structures, mixer-settler type equipment equipped with internal mixing elements, and extraction devices utilizing centrifugal force can be used.

As a working example of the second new extraction process, a batch extraction device was used wherein the material was extracted with liquid carbon dioxide. Drums containing 80 kg of degummed resin from Boswellia serrata were charged into a suitable extraction chamber and contacted with liquid carbon dioxide for 2 hours. Each 80 kg charge yielded at least 18 kg of an enriched pasty material containing boswellic acids and other organic acids.

Also within the scope of the present invention is an extract obtained from Boswellia serrata obtained with one of the new extraction processes of the present invention. For instance, a total organic acids extract from Boswellia serrata can be obtained with the first or second new extraction process of the present invention.

### REFERENCES

1. Ammon, H. P. T. (1993) Application of pure boswellic acids. Patent No. 0 552 657 A1. European Patent Office.
2. Ammon, H. P. T. (1997) Use of Boswellic acids and its derivatives for inhibiting normal and increased leucocytic elastase or plasmin activity. Patent WO 97/07796. European Patent Office.
3. Mukherji, S. et al. (1970) Studies on plant anti-tumor agents. Ind J Pharm 32:48.
4. Lee. Yue-Wei (1991) Pentacyclic triterpenoid compounds as topoisomerase inhibitors or cell
differentiation inducers. U.S. Pat. No. 5,064,823.
5. Safayhi, H. et al. (1992) Boswellic acids: novel, specific, non-redox inhibitors of 5-lipoxygenase. J. Pharmacol. Exp. Ther. 261:1143-6.
6. Safayhi. H. et al. (1997) Inhibition by boswellic acids of human leukocyte elastase. J. Pharmacol. Err Ther. 281:460-463.

1-85. (canceled)

86. A method for the treatment of leukemia, comprising administering an effective amount of a composition comprising boswellic acids consisting essentially of
β-boswellic acid of at least 12% by weight;
acetyl-β-boswellic acid of at least 5% by weight;
11-keto-β-boswellic acid of at least 1% by weight; and
acetyl-11-keto-β-boswellic acid of at least 1% by weight.

87. The method of claim 86, wherein the boswellic acids consist essentially of
β-boswellic acid of at least 12% by weight;
acetyl-β-boswellic acid of at least 10% by weight;
11-keto-β-boswellic acid of at least 5% by weight; and
acetyl-11-keto-β-boswellic acid of at least 4% by weight.

88. The method of claim 87, wherein the boswellic acids consist essentially of
β-boswellic acid of 12 to 35% by weight;
acetyl-β-boswellic acid of 10 to 35% by weight;
11-keto-β-boswellic acid of 5 to 45% by weight; and
acetyl-11-keto-β-boswellic acid of 5 to 45% by weight.

89. The method of claim 86, wherein the composition is administered to a human or an animal.

90. The method of claim 89, wherein the composition is administered to a human.

91. The method of claim 86, wherein the leukemia is selected from the group consisting of myeloid leukemia, acute myelogenous leukemia, acute lymphocytic leukemia, acute non-lymphocytic leukemia, chronic lymphocytic leukemia and hairy cell leukemia.
